(19) United States
(12) Patent Application Publication
(10) Pub. No.: US 2006/0140883 A1
(43) Pub. Date: Jun. 29, 2006

(54) Oral care compositions containing a eucalyptus extract

(75) Inventors: Harsh M. Trivedi, Somerset, NJ (US); Tao Xu, East Brunswick, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/239,186

(22) Filed: Sep. 29, 2005

Related U.S. Application Data
(60) Provisional application No. 60/640,175, filed on Dec. 29, 2004.

(51) Int. Cl. A61K 8/97; A61K 36/61

(52) U.S. Cl. 424/58; 424/742

(57) ABSTRACT

Oral care compositions comprising an antibacterial effective amount of an antibacterial system comprising a substantially water insoluble, noncationic antibacterial agent and Eucalyptus extract and an orally acceptable carrier. Methods of inhibiting oral bacteria in the oral cavity of a human or other animal subject are also provided.

### CROSS REFERENCE TO RELATED APPLICATION

This application claims the benefit of U.S. Ser. No. 60/640,175, filed Dec. 29, 2004, the contents of which are incorporated herein by reference.

### BACKGROUND OF THE INVENTION

Dental plaque is a by-product of microbial growth and comprises a layer of microorganisms in a polysaccharide matrix. Plaque is a soft deposit which forms on teeth and may form on any part of the tooth surface, particularly at the gingival margin. Even when following a dental hygiene program, plaque reforms and may build up on dental surfaces, especially in recessed areas of tooth surfaces. Hence, beside being unsightly, it is implicated in the occurrence of dental caries, dental calculus and gingivitis. Gingivitis, if untreated, could lead to other complications such as periodontitis and eventually loss of teeth.

Many measures, in addition to regular tooth brushings have been proposed as a means of removing dental plaque thereby reducing plaque and carries causing bacteria. These include, for example, using dentifrices or mouthwashes containing antibacterial/antiplaque agents formulated to retard plaque formation and the oral infections associated with plaque formation. However, most antibacterial agents are incompatible or unstable with other oral care active ingredients, or may inactivate such oral care ingredients. Therefore, it is desirable to form an oral care composition that prevents or combats plaque formation, as well as other detrimental oral care symptoms.

### BRIEF SUMMARY OF THE INVENTION

It has been discovered that compositions and methods of this invention afford advantages over antibacterial and antiplaque compositions among those known in the art. Such advantages include providing an oral care composition that is stable and highly effective as an antibacterial/antiplaque treatment. Further, the oral composition comprises an antibacterial active ingredient that is natural and derived from a botanical source. Further uses, benefits and embodiments of the present invention are apparent from the description set forth herein.

### DETAILED DESCRIPTION OF THE INVENTION

Compositions of the present invention comprise an extract of Eucalyptus. “Eucalyptus extract(s)” as used herein are complex mixtures of different organic molecules obtained from, such as Eucalyptus terpenes, Eucalyptus alcohols, Eucalyptus esters, Eucalyptus aldehydes, Eucalyptus ketones, and Eucalyptus phenols from a Eucalyptus plant, shrub, or tree, or a synthetic or semi-synthetic equivalent of such an extract.

The Eucalyptus extract may be derived from any species of Eucalyptus. Exemplary Eucalyptus extracts are derived from Eucalyptus grandis, Eucalyptus botryoides, Eucalyptus globulus, Eucalyptus maculata, Eucalyputs viminalis, Eucalyptus camaldulensis, and Eucalyptus crebra. Preferably, Eucalyptus globulus is the source of the extract.

The Eucalyptus extract can be derived from at least one Eucalyptus tissue selected from the group comprising leaves, stems, buds, flowers, roots, and bark. The chemical composition of the extract may be tailored by using particular combinations of tissues or with planned tissue harvesting or treatment. Preferably, at least a portion of the extract is obtained from a leaf. However, it is understood that the foliage or any other tissues may come from a single tree, plant, or shrub or multiple trees, plants, or shrubs within the same or different species.

The extract may be removed from the Eucalyptus tissue using a variety of techniques known or to be developed in the art. Suitable techniques include those disclosed in U.S. Pat. No. 6,352,727, Takahashi, issued Mar. 5, 2002.

An example polar Eucalyptus extract may include eucalyptol and dihydrochalchone represented by the following formula (I); and including

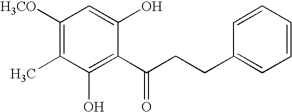

The Eucalyptus extract comprises from about 0.001% to about 5% of the composition, preferably no more than about 0.02% to about 0.3%.

The composition of the invention may also contain an additional antibacterial agent. The antibacterial agent may be a substantially water insoluble, noncationic antibacterial agent, preferably an alkylphenoxy phenol; a cycloalkyl-phenoxyphenol; a 9,10-dihydrophenanthrenol; an alkylphenol; a cycloalkyl-phenol; a phenolic compound; a halogenated carbanilide; a halogenated salicylanilide; a benzoic ester; a halogenated diphenyl ether, and mixtures thereof.

The substantially water insoluble, noncationic, antibacterial alkylphenoxy phenol or cycloalkyl-phenoxyphenol or -9,10-dihydrophenanthrenol can include a substantially water insoluble, noncationic antibacterial phenol containing, relative to the hydroxyl group, an alkyl or cycloalkyl group, preferably tert-butyl (t-butyl), in 2-position, and substituents in one or both of the 4- and 5-positions, one of which may be phenyl or 2′, 3′ and/or 4′ substituted alkyl or cycloalkyl phenyl, preferably 4′-t-butyl phenyl or a phenanthrene containing a hydroxyl substituent in the 2- or 3-position and alkyl or cycloalkyl, preferably t-butyl, substituents in the other of the 2- and 3-positions and in at least one of the other rings and are described in U.S. Pat. No. 5,723,500, the contents of which are incorporated herein by reference.

The water insoluble, noncationic, antibacterial alkyl-phenol or cycloalkyl-phenol include a phenol containing, relative to the hydroxyl group, an alkyl or cycloalkyl group, preferably tert-butyl (t-butyl), in the 2-position, and substituents in one or both of the 4- and 5-positions, one or both of which may be alkyl or cycloalkyl, one being preferably t-butyl, such as those described in U.S. Pat. No. 5,912,274, the contents of which are incorporated herein by reference.

The phenolic compounds among those useful herein include phenol and its homologs, mono and polyalkyl and aromatic halophenols, resorcinol and its derivatives, and bisphenolic compounds, such as those disclosed in U.S. Pat. No. 5,368,844, Gaffar et al., the contents of which are incorporated herein by reference. Preferred phenolic compounds are n-hexyl resorcinol and 2,2′-methylene bis (4-chloro-6-bromophenol).

Exemplary halogenated carbanilides, halogenated salicylanilides and benzoic esters are disclosed in U.S. Pat. No. 5,776,435, Gaffar et al., the contents of which are incorporated herein by reference. Halogenated carbanilides include 3,4,4′-trichlorocarbanilide, 3-trifluoromethyl-4,4′-dichlorocarbanilide, and 3,3′,4-trichlorocarbanilide. Halogenated salicylanilides include 4′5-dibromosalicylanilide, 3,4′,5-trichlorosalcylanilide, 3,4′,5-tribromosalicylanilide, 2,3,3′,5-tetrachlorosalicylanilide, 3,3′,5-tetrachlorosalicylanilide, 3,5-dibromo-3′-trifluoromethyl salicylanilide, 5-n-octanoyl-3′-trifluoromethyl salicylanilide, 3,5-dibromo-4′-trifluoromethyl salicylanilide, 3,5-dibromo-3′-trifluoro methyl salicylanilide (Fluorophene), and mixtures thereof. Benzoic esters include methyl-p-hydroxybenzoic ester, ethyl-p-hydroxybenzoic ester, propyl-p-hydroxybenzoic ester, and butyl-p-hydroxybenzoic ester.

Preferably, the antibacterial agent is a substantially water insoluble, noncationic diphenyl ether selected from the group comprising 2,4,4′-trichloro-2′-hydroxydiphenyl ether (triclosan) and 2,2′-dihydroxy-5,5′-dibromodiphenyl ether, and is preferably triclosan.

The substantially water insoluble, noncationic antibacterial agent is present in the composition at about 0.001% to about 5%. Preferably, the antibacterial agent is present in the composition at no more than about 0.02% to about 0.3%.

The present invention comprises the substantially water insoluble, noncationic antibacterial agent and the Eucalyptus extract in a system which is preferably highly efficacious in plaque removal and preventing bacterial accumulation. In preferred embodiments, the “system” comprises components that, when combined together, provide antibacterial activity. Preferably the efficacy of the system in a composition of the present invention is greater than the efficacy that would be afforded by either component of the system by itself.

In various embodiments, the antibacterial system may comprise low levels, about 0.0001% to about 0.3%, preferably about 0.02% to about 0.05%, of each of the substantially water insoluble, noncationic antibacterial agent and the Eucalyptus components and show a Minimal Inhibitory Concentration (MIC) far less than the MIC of the same amount of either components alone or in combination. While not intending to be bound by any one particular theory, the surprisingly efficacious antibacterial system effect is not believed to be cumulative.

In various embodiments, the antibacterial system further comprises at least one of a solubilizing agent, an antibacterial enhancing agent, a fluoride providing agent, and an anticalculus agent. Solubilizing agents include the humectant polyols such as propylene glycol, dipropylene glycol, and hexylene glycol, cellosolves such as methyl cellosolve and ethyl cellosolve, vegetable oils and waxes containing at least about 12 carbon atoms in a straight chain such as olive oil, castor oil, and petrolatum and esters such as amyl acetate, ethyl acetate, glyceryl tristearate, and benzyl benzoate. Propylene glycol is preferred. As used herein, “propylene glycol” includes 1,2-propylene glycol and 1,3-propylons glycol. Additional suitable solubilizing agents include surfactants, non-toxic alcohols, and flavoring oils. Exemplary solubilizing agents are described in U.S. Pat. No. 4,894,220, Nabi et al., U.S. Pat. No. 5,605,676, and U.S. Pat. No. 5,728,756, the contents of each which are incorporated herein by reference.

In embodiments comprising a halogenated diphenyl ether, it is preferred that the composition, including the solubilizing agent, carrier components, and optional ingredients, such as those described later herein, is essentially free from polyethylene glycol. The solubilizing agent is present in the composition in a sufficient amount to assist dissolving the antibacterial agent in saliva, preferably at about 0.02% to about 50% or from about 0.02% to about 2%.

The antibacterial enhancing agent is a water soluble or swellable anionic polymer or copolymer comprising at least one delivery-enhancing functional group and at least one organic retention-enhancing group, wherein said delivery-enhancing group enhances delivery of said antibacterial system to oral tooth and gum surfaces and said retention-enhancing group enhances attachment adherence or bonding of said antibacterial system on oral tooth and gum surfaces. Preferably, the antibacterial enhancing agent is a anionic polymeric polycarboxylate or salt thereof, or phosphonate polymer or salt thereof having an average molecular weight of about 100 to about 1,000,000. In an embodiment comprising a polymeric polycarboxylate, the salt may be an alkali metal or ammonium salt. The polymeric polycarboxylate may be a synthetic anionic polymeric polycarboxylate having an average molecular weight of about 1,000 to about 1,000,000. In various embodiments, the polymeric polycarboxylate is an anionic copolymer of maleic acid or anhydride with at least one further ethylenically unsaturated polymerizable monomer, preferably a copolymer of maleic acid or anhydride with methyl vinyl ether, which is commercially available as GANTREZ® (International Specialty Products, Wayne, N.J., USA). Suitable methyl vinyl ethers include GANTREZ® AN (MW 200,000 to 2,000,000), S (MW 700,000 to 1,500,000), MS (MW 1,000,000), and ES (MW 90,000 to 150,000). Preferably, the antibacterial enhancing agent is present in the composition at about 0.0005% to about 5%.

In an embodiment comprising a phosphonate polymer, the phosphonate polymer salt is an alkali metal or ammonium salt. The phosphonate polymer may be a polyvinyl phosphonate, poly(α-styrene phosphonate), poly(β-styrene phosphonate), or copoly (α-,β-styrene phosphonate). The phosphonate polymer may also be a copolymer of α- or β-styrene phosphonate with another polymerizable ethylenically unsaturated monomer, preferably copoly (β-styrenephosphonate/vinylphosphonate). The phosphonate polymer may have an average molecular weight of about 100 to about 1,000,000, preferably 2,000 to about 30,000.

In another embodiment the composition comprises an orally acceptable source of fluoride ions. One or more such sources can be present. Suitable sources of fluoride ions include fluoride, monofluorophosphate and fluorosilicate salts. Any such salt that is orally acceptable can be used, including without limitation alkali metal (e.g., potassium, sodium), ammonium, stannous and indium salts and the like. Amine fluorides, including olaflur (N′-octadecyltrimethylendiamine-N,N,N′-tris(2-ethanol)-dihydrofluoride) may also be used. Water-soluble fluoride-releasing salts are typically used. One or more fluoride-releasing salts are optionally present in an amount providing a total of about 100 to about 20,000 ppm, about 200 to about 5,000 ppm, or about 500 to about 2,500 ppm, fluoride ions. Where sodium fluoride is the sole fluoride-releasing salt present, illustratively an amount of about 0.01% to about 5%, about 0.05% to about 1% or about 0.1% to about 0.5%, sodium fluoride by weight can be present in the composition.

In an embodiment comprising an anti-calculus agent, one or more such agents can be present. Suitable anticalculus agents include without limitation phosphates and polyphosphates (for example pyrophosphates), polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, disodium dihydrogen pyrophosphate, sodium trimetaphosphate, sodium hexametaphosphate and the like, wherein sodium can optionally be replaced by potassium or ammonium.

In various embodiments, the orally acceptable dentifrice vehicle used to prepare the dentifrice composition comprises a water-phase. As recognized by one of skill in the art, the oral compositions of the present invention optionally include other materials, such as for example, anticaries agents, desensitizing agents, viscosity modifiers, diluents, surface active agents, such as surfactants, emulsifiers, and foam modulators, pH modifying agents, abrasives, humectants, mouth feel agents, sweetening agents, flavor agents, colorants, preservatives, and combinations thereof. It is understood that while general attributes of each of the above categories of materials may differ, there may be some common attributes and any given material may serve multiple purposes within two or more of such categories of materials. Preferably, such carrier materials are selected for compatibility with the antibacterial system, as well as with other ingredients of the composition. The composition may take any form desired for an oral care product, including pate, gel, mouthrinse, pastille, confectionary forms, tablets, films, tapes, lozenges or beads.

In various embodiments of the present invention, glycerin, propylene glycol, sorbitol, polypropylene glycol and/or polyethylene glycol (e.g., 400-600) are suitable humectants/carriers. Also advantageous are liquid mixtures of water, glycerin, and sorbitol. In certain embodiments where the carrier is a clear gel and where the refractive index is an important consideration, the composition comprises about 3 to about 30% of water, 0 to about 70% of glycerin and about 20 to about 80% of sorbitol. In a preferred embodiment, the oral composition will be substantially free of polyethylene glycol, preferably, the composition will contain no polyethylene glycol.

In various embodiments, toothpastes, creams and gels contain a natural or synthetic thickener or gelling agent, which, other than silica thickeners, include natural and synthetic gums and colloids. In a still further embodiment a composition of the invention comprises at least one thickening agent, useful for example to impart a desired consistency and/or mouth feel to the composition. Any orally acceptable thickening agent can be used, including without limitation carbomers, also known as carboxyvinyl polymers, carrageenans, also known as Irish moss and more particularly ι-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. One or more thickening agents are optionally present in a total amount of about 0.01% to about 15%, for example about 0.1% to about 10% or about 0.2% to about 5% by weight of the composition.

Various embodiments of the present invention also comprise a surface active agent, which may function as a surfactant, emulsifier, and/or foam modulator. Surface active agents generally achieve increased prophylactic action, by thoroughly dispersing the antibacterial system throughout the oral cavity. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01% to about 10%, for example about 0.05% to about 5% or about 0.1% to about 2% by weight of the composition.

In various embodiments of the present invention, where the vehicle of the oral care composition is solid or a paste, the oral composition preferably comprises a dentally acceptable abrasive material or polishing agent, which may serve to either polish the tooth enamel or provide a whitening effect. Any orally acceptable abrasive can be used, but type, fineness (particle size) and amount of abrasive should be selected so that tooth enamel is not excessively abraded in normal use of the composition. Suitable abrasives include without limitation silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. One or more abrasives are optionally present in an abrasive effective total amount, typically about 5% to about 70%, for example about 10% to about 50% or about 15% to about 30% by weight of the composition. Average particle size of an abrasive, if present, is generally about 0.1 to about 30 μm, for example about 1 to about 20 μm or about 5 to about 15 μm.

In various embodiments of the present invention, water is also present in the oral composition, as referred to above. Water employed in the preparation of commercially suitable toothpastes, gels, and mouthwashes should preferably be deionized and free of organic impurities. The water is free water which is added, plus that which is introduced with other materials for example, such as that added with sorbitol. Water generally comprises from about 10% to 50%, preferably from about 20% to 40%, of the toothpaste compositions herein. Water is a preferred diluent and in some compositions such as mouthwashes and whitening liquids is commonly accompanied by an alcohol, e.g., ethanol. The weight ratio of water to alcohol in a mouthwash composition is generally about 1:1 to about 20:1, for example about 3:1 to about 20:1 or about 4:1 to about 10:1.

Flavorants among those useful herein include any material or mixture of materials operable to enhance the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include methol, menthyl acetate, menthyl lactate, camphor, Eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof. One or more flavorants are optionally present in a total amount of about 0.01% to about 5%, optionally in various embodiments from about 0.05 to about 2%, from about 0.1% to about 2.5%, and from about 0.1 to about 0.5%.

Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof. One or more sweeteners are optionally present in a total amount depending strongly on the particular sweetener(s) selected, but typically at levels of from about 0.005% to about 5%, optionally from about 0.01% to about 1%.

Colorants among those useful herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5%.

Humectants useful herein include polyhydric alcohols such as glycerin, sorbitol, xylitol and low molecular weight polyethylene glycols, including those listed above herein. In various embodiments, humectants are operable to prevent hardening of paste or gel compositions upon exposure to air. In various embodiments humectants also function as sweeteners. One or more humectants are optionally present in a total amount of about 1% to about 50%, for example about 2% to about 25% or about 5% to about 15%.

pH modifying agents among those useful herein include acidifying agents to lower pH, basifying agents to raise pH, and buffering agents to control pH within a desired range. For example, one or more compounds selected from acidifying, basifying, and buffering agents can be included to provide a pH of about 2 to about 10, or in various embodiments from about 2 to about 8, from about 3 to about 9, from about 4 to about 8, from about 5 to about 7, from about 6 to about 10, and from about 7 to about 9. Any orally acceptable pH modifying agent can be used, including carboxylic, phosphoric, and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole, and mixtures thereof. One or more pH modifying agents are optionally present in a total amount effective to maintain the composition in an orally acceptable pH range.

Mouth-feel agents that may be used herein include materials which impart a desirable texture or other feeling during use of the composition. Such agents include bicarbonate salts, which in various embodiments impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate, and mixtures thereof. One or more bicarbonate salts are optionally present in a total amount of 0.1% to about 50%, for example about 1% to about 20%.

The compositions of the present invention may optionally comprise a stannous ion source useful, for example, in helping reduce gingivitis, plaque, calculus, caries or sensitivity. One or more such sources can be present. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5% by weight of the composition.

The compositions comprise an orally acceptable antimicrobial (e.g., antibacterial) agent other than the Eucalyptus extract or substantially water insoluble noncationic antibacterial agent as described above. One or more such agents can be present. Suitable examples include without limitation copper (II) compounds such as copper (II) chloride, fluoride, sulfate and hydroxide, zinc ion sources such as zinc acetate, zinc citrate, zinc gluconate, zinc glycinate, zinc oxide, zinc sulfate and sodium zinc citrate, phthalic acid and salts thereof such as magnesium monopotassium phthalate, hexetidine, octenidine, sanguinarine, benzalkonium chloride, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, iodine, sulfonamides, bisbiguanides such as alexidine, chlorhexidine and chlorhexidine digluconate, piperidino derivatives such as delmopinol and octapinol, magnolia extract, grapeseed extract, menthol, geraniol, citral, eucalyptol, antibiotics such as augmentin, amoxicillin, tetracycline, doxycycline, minocycline, metronidazole, neomycin, kanamycin and clindamycin, and the like. A further illustrative list of useful antibacterial agents is provided in U.S. Pat. No. 5,776,435 to Gaffar et al., the contents of which are incorporated herein by reference. One or more antimicrobial agents other than phenolic compounds are optionally present in an antimicrobial effective total amount, typically about 0.05% to about 10%, for example about 0.1% to about 3% by weight, of the composition.

The compositions of the present invention optionally comprise an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

The compositions of the present invention optionally comprise a sialogogue or saliva-stimulating agent, useful for example in amelioration of dry mouth. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

The compositions of the present invention optionally comprise an orally acceptable zinc ion source useful, for example, as an antimicrobial, anticalculus or breath-freshening agent. One or more such sources can be present. Suitable zinc ion sources include without limitation zinc acetate, zinc citrate, zinc gluconate, zinc glycinate, zinc oxide, zinc sulfate, sodium zinc citrate and the like. One or more zinc ion sources are optionally and illustratively present in a total amount of about 0.05% to about 3%, for example about 0.1% to about 1%, by weight of the composition.

The compositions of the present invention optionally comprise an antiplaque (e.g., plaque disrupting) agent. One or more such agents can be present in an antiplaque effective total amount. Suitable antiplaque agents include without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and chelating agents such as citric and tartaric acids and alkali metal salts thereof.

The compositions of the present invention optionally comprise an anti-inflammatory agent. One or more such agents can be present in an anti-inflammatory effective total amount. Suitable anti-inflammatory agents include without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in the composition in an anti-inflammatory effective amount.

The compositions of the present invention optionally comprise an H₂ histamine receptor antagonist. H2 antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

The compositions of the present invention optionally comprise a desensitizing, or tooth sensitivity protecting, agent. One or more such agents can be present. Suitable desensitizing agents include without limitation potassium salts such as potassium citrate, potassium tartrate, potassium chloride, potassium sulfate and potassium nitrate. Another suitable desensitizing agent is sodium nitrate. Alternatively or in addition a local or systemic analgesic such as aspirin, codeine, acetaminophen, sodium salicylate or triethanolamine salicylate can be used. One or more desensitizing agents and/or analgesics are optionally present in a desensitizing and/or analgesic effective amount, typically about 0.05% to about 5%, for example about 0.1% to about 3% by weight, of the composition.

The compositions of the present invention optionally comprise a nutrient. Suitable nutrients include vitamins, minerals, amino acids, and mixtures thereof. Vitamins include vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), fish oil (including components thereof such as omega-3 (N-3) polyunsaturated fatty acids, eicosapentaenoic acid and docosahexaenoic acid), coenzyme Q10, and mixtures thereof.

The compositions of the present invention optionally comprise proteins. Suitable proteins include milk proteins and enzymes such as peroxide-producing enzymes, amylase, plaque-disrupting agents such as papain, glucoamylase, and glucose oxidase.

In various embodiments, the present invention provides a method of inhibiting bacteria in the oral cavity of a human or other animal subject, where the method comprises administering to the oral cavity an antibacterial system comprising an Eucalyptus extract and a substantially water insoluble, noncationic antibacterial agent. The oral care composition is contacted with the oral surface of the mammalian subject to thereby kill bacteria and reduce both plaque formation and calculus formation in a highly efficacious manner, without any negative interaction between the antibacterial system and the orally acceptable vehicle.

In various embodiments, it is preferred that the oral care composition is applied and contacted with the oral surface. The dentifrice, confectionery, or mouthwash prepared in accordance with the present invention is preferably applied regularly to a oral surface, preferably on a daily basis, at least one time daily for multiple days, but alternately every second or third day. Preferably the oral composition is applied to the oral surfaces from 1 to 3 times daily, at a pH of about 4.5 to about 9, generally about 5.5 to about 8, preferably about 6 to 8, for at least 2 weeks up to 8 weeks, or more up to lifetime.

The oral compositions of the present invention may be prepared by suitably mixing the ingredients. For instance, in the preparation of a mouthrinse, the antibacterial system comprising Eucalyptus extract and the substantially water insoluble, non-cationic antibacterial agent are dispersed in a mixture of ingredients, e.g., alcohol, humectants, surfactants, and flavor are then added and mixed. The ingredients are then mixed under vacuum for about 15-30 minutes. The resulting rinse product is then packaged. Dentifrices are prepared similarly, additional thickener and abrasives agents being included in the last step.

Embodiments of the present invention may be made by providing at least one Eucalyptus extract, at least one substantially water insoluble, noncationic antibacterial agent and an orally acceptable carrier. These components and optional components, including, but not limited to solubilizing agent, an antibacterial enhancing agent, a fluoride providing agent, and an anticalculus agent are mixed to form a toothpaste or gel. In an embodiment comprising a dentifrice, it may be desirable to completely homogenize the composition or it may be desirable to partially homogenize the mixture depending on the presence of additional components (e.g., dental film strips containing an oral care active, e.g., breath strips, such as those described in U.S. Pat. No. 6,669,929, Boyd et al., issued Dec. 30, 2003) in the composition.

The antiplaque, antibacterial, anticalculus oral composition of this invention can be incorporated into a confectionery or troche. Such methods of forming confectionery (e.g., gum) or troches (e.g., lozenges) are well known by one of skill in the art, and can be prepared by stirring into a warm gum base or coating the outer surface of a gum base (for example, jelutone, rubber latex, vinylite resins, inter alia), desirably with conventional plasticizers or softeners, sugar or other sweeteners or carbohydrates such as glucose, sorbitol and the like.

The present invention is further illustrated through the following non-limiting examples.

### EXAMPLES

### Example 1

A glycerin/sorbitol-based toothpaste composition is prepared having ingredients as shown in the following table. The Eucalyptus globulus extract is present at 0.2% by weight of the composition. Triclosan is present at 0.2% by weight of the composition. The antibacterial system reduces dental plaque and provides enhanced antibacterial efficacy.

| Ingredient | Weight Percent |
|---|---|
| Toothpaste base¹ | 99.6 |
| Eucalyptus globulus extract | 0.2 |
| Triclosan | 0.2 |
| Total | 100 |

¹toothpaste base includes the following ingredients: silica powder, glycerin, sorbitol, water, Gantrez ®, sodium lauryl sulfate, sodium hydroxide, titanium dioxide, flavor, sodium CMC, ι-carrageenan, sodium fluoride, and sodium saccharin.

### Example 2

A sorbitol-based toothpaste containing silica abrasives, a PVME/MA (Gantrez) anticalculus agent, sodium fluoride and other conventional ingredients is prepared according to the following table. The Eucalyptus globulus extract is present at 0.09% by weight of the composition. Triclosan is present at 0.18% by weight of the composition. The antibacterial toothpaste is effective to reduce oral bacteria and dental plaque.

| Ingredient | Weight Percent |
|---|---|
| Toothpaste base² | 99.73 |
| Eucalyptus globulus extract | 0.09 |
| Triclosan | 0.18 |
| Total | 100 |

²toothpaste base includes the following ingredients: sorbitol, water, silica powder, Gantrez ®, sodium lauryl sulfate, sodium hydroxide, titanium dioxide, flavor, ι-carrageenan, glycerin, sodium fluoride, and sodium saccharin.

### Example 3

A toothpaste composition is prepared according to Example 2. Eucalyptus viminalis is substituted for the Eucalyptus globulus in the antibacterial system, with substantially similar results.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. An oral care composition, comprising:
a safe and effective antibacterial amount of an antibacterial system comprising:
a Eucalyptus extract; and
a substantially water insoluble, noncationic antibacterial agent, and
an orally acceptable carrier.

2. An oral care composition according to claim 1, wherein said substantially water insoluble, noncationic antibacterial agent comprises an agent selected from the group consisting of an alkylphenoxy phenol; a cycloalkyl-phenoxyphenol; a 9,10-dihydrophenanthrenol; an alkylphenol; a cycloalkyl-phenol; a phenolic compound; a halogenated carbanilide; a halogenated salicylanilide; a benzoic ester; a halogenated diphenyl ether, and mixtures thereof.

3. An oral care composition according to claim 2, wherein said substantially water insoluble, noncationic antibacterial agent is a halogenated diphenyl ether.

4. An oral care composition according to claim 3, wherein said halogenated diphenyl ether is 2,4,4′-trichloro-2′-hydroxydiphenyl ether.

5. An oral care composition according to claim 1, wherein said antibacterial agent is present in the composition at about 0.001% to about 5%.

6. An oral care composition according to claim 5, wherein said antibacterial agent is present in the composition at about 0.02% to about 0.3%.

7. An oral care composition according to claim 1, wherein said Eucalyptus extract is from tissues derived of at least one of Eucalyptus grandis, Eucalyptus botryoides, Eucalyptus globulus, Eucalyptus maculata, Eucalyputs viminalis, Eucalyptus camaldulensis, and Eucalyptus crebra.

8. An oral care composition according to claim 7, wherein said Eucalyptus extract is derived from Eucalyptus globulus tissues.

9. An oral care composition according to claim 7, wherein said Eucalyptus extract is derived from at least one Eucalyptus tissue selected from the group consisting of leaves, stems, buds, flowers, roots, and bark.

10. An oral care composition according to claim 9, wherein said Eucalyptus tissue comprises a Eucalyptus leaf tissue.

11. An oral care composition according to claim 9, wherein said Eucalyptus tissue is at least partially dehydrated.

12. An oral care composition according to claim 7, wherein said Eucalyptus extract comprises at least one of an Eucalyptus terpene, an Eucalyptus alcohol, an Eucalyptus ester, an Eucalyptus aldehyde, an Eucalyptus ketone, and an Eucalyptus phenolic compound.

13. An oral care composition according to claim 12, wherein said Eucalyptus phenolic compound is a dihydrochalcone.

14. An oral care composition according to claim 1, wherein said composition comprises from about 0.001% to about 5% of said Eucalyptus extract.

15. An oral care composition according to claim 14, wherein said composition comprises from about 0.02% to about 0.3% of said Eucalyptus extract.

16. An oral care composition according to claim 1, further comprising at least one of a solubilizing agent, an antibacterial enhancing agent, a fluoride providing agent, and an anticalculus agent.

17. An oral care composition according to claim 16, wherein said solubilizing agent is selected from the group consisting of: a humectant polyol; a cellosolve; a vegetable oil or wax containing at least 12 carbon atoms in a straight chain; an ester; a surfactant; non-toxic alcohols; flavoring oils; and mixtures thereof.

18. An oral care composition according to claim 16, wherein said solubilizing agent is present at about 0.02% to about 50% by weight.

19. An oral care composition according to claim 16, wherein said antibacterial enhancing agent is a water soluble or swellable anionic polymer or copolymer comprising at least one delivery-enhancing functional group and at least one organic retention-enhancing group, wherein said delivery-enhancing group enhances delivery of said antibacterial system to oral tooth and gum surfaces and said retention-enhancing group enhances attachment adherence or bonding of said antibacterial system on oral tooth and gum surfaces.

20. An oral care composition according to claim 19, wherein said antibacterial enhancing agent is a polymeric polycarboxylate or salt thereof or phosphonate polymer or salt thereof having an average molecular weight of about 100 to about 1,000,000.

21. An oral care composition according to claim 20, wherein said polymeric polycarboxylate is a synthetic anionic polymeric polycarboxylate.

22. An oral care composition according to claim 21, wherein said polymeric polycarboxylate is an anionic copolymer of maleic acid or anhydride with at least one further ethylenically unsaturated polymerizable monomer having an average molecular weight of about 1,000 to about 1,000,000.

23. An oral care composition according to claim 20, wherein said antibacterial enhancing agent is a phosphonate polymer or salt thereof.

24. An oral care composition according to claim 23, wherein said polymeric phosphonate is a polyvinyl phosphonate, poly(α-styrene phosphonate), poly(β-styrene phosphonate), copoly(α-,β-styrene phosphonate), or a copolymer of α- or β-styrene phosphonate with another polymerizable ethylenically unsaturated monomer.

25. An oral care composition according to claim 16, wherein said antibacterial enhancing agent is present in the composition at about 0.0005% to about 5%.

26. An oral care composition according to claim 16, wherein said anticalculus agent is a polyphosphate.

27. An oral care composition according to claim 1, wherein said composition is a dentifrice.

28. A method of inhibiting bacteria in the oral cavity of a human or other animal subject, comprising administering to said oral cavity a safe and effective antibacterial amount of an antibacterial system comprising:
a Eucalyptus extract; and
a substantially water insoluble, noncationic antibacterial agent, wherein said antibacterial system is in an orally acceptable carrier.

29. A method according to claim 28, wherein said administering further comprises applying said antibacterial system at least once daily.

30. A method according to claim 28, wherein said substantially water insoluble, noncationic antibacterial agent comprises at least one of: an agent selected from the group consisting of an alkylphenoxy phenol; a cycloalkyl-phenoxyphenol; a 9,10-dihydrophenanthrenol; an alkylphenol; a cycloalkyl-phenol; a phenolic compound; a halogenated carbanilide; a halogenated salicylanilide; a benzoic ester; a halogenated diphenyl ether, and mixtures thereof.

31. A method according to claim 30, wherein said halogenated diphenyl ether is 2,4,4′-trichloro-2′-hydroxydiphenyl ether.

32. A method according to claim 28, wherein said Eucalyptus extract is derived from at least one of Eucalyptus grandis, Eucalyptus botryoides, Eucalyptus globulus, Eucalyptus maculata, Eucalyputs viminalis, Eucalyptus camaldulensis, and Eucalyptus crebra.

33. A method according to claim 32, wherein said Eucalyptus extract is derived from Eucalyptus globulus.

34. A method according to claim 28, wherein said composition further comprises at least one of a solubilizing agent, an antibacterial enhancing agent, a fluoride providing agent, and an anticalculus agent.

35. An oral care composition, comprising:
a safe and effective antibacterial amount of an antibacterial system, comprising:
a Eucalyptus globulus extract; and
a halogenated diphenyl ether antibacterial agent;
an antibacterial enhancing agent;
a solubilizing agent;
a fluoride providing agent;
an anti-calculus agent; and
an orally acceptable carrier.
